(19) United States
(12) Patent Application Publication
(10) Pub. No.: US 2025/0375409 A1
(43) Pub. Date: Dec. 11, 2025

(54) Treatment of REM Behavior Disorder

(71) Applicant: Vanda Pharmaceuticals Inc., Washington, DC (US)

(72) Inventor: Mihael Polymeropoulos, Washington, DC (US)

(21) Appl. No.: 19/234,687

(22) Filed: Jun. 11, 2025

Related U.S. Application Data
(60) Provisional application No. 63/658,527, filed on Jun. 11, 2024.

(51) Int. Cl. A61K 31/343

(52) U.S. Cl. CPC A61K 31/343

(57) ABSTRACT

Embodiments of the invention relate generally to the treatment of sleep-related disorders. One aspect of the invention includes a method of treating a patient suffering from rapid eye movement (REM) behavior disorder (RBD) comprising: administering to the patient an effective dose of tasimelteon before bedtime.

### CROSS-REFERENCE TO RELATED APPLICATIONS

This application claims the benefit of co-pending U.S. Provisional Patent Application Ser. No. 63/658,527, filed 11 Jun. 2024, which is incorporated herein as though fully set forth.

### BACKGROUND

### REM Behavior Disorder (RBD)

Rapid eye movement (REM) behavior disorder (RBD) is characterized by abnormal behaviors that emerge from REM sleep and can lead to injury and disturbed sleep. Most patients have frequent events, typically more than once per week. Abnormalities can be seen almost nightly and consist of intermittent loss of the normal muscle atonia of REM sleep. This phenomenon is used as a diagnostic criterion even in the absence of an overt clinical event during the night.

RBD has serious consequences for the health of the patient. Aside from a risk of sometimes severe injury, a direct consequence of a violent nocturnal movement, it often leads to sleep disruption. Furthermore, it is commonly seen in association with Parkinson's disease and many experts in the field consider it a prodrome of neurodegenerative conditions. Other comorbidities may include higher risks of cerebral hemorrhage and stroke. Multiple factors may contribute to the risk of RBD. Aside from neurodegenerative conditions, RBD is seen in association with disorders of REM sleep regulations, including narcolepsy and post-traumatic stress disorder as well as with use of selective serotonin reuptake inhibitors (SSRIs).

In healthy individuals, REM sleep is closely linked to circadian phase, with a peak a little after the nadir of the core body temperature and thus also around the time when melatonin secretion is maximal. Studies using a forced desynchrony protocol suggest that the circadian system has a primary effect of REM sleep regulation with a modifying effect from the homeostatic factors. Various other factors affect REM sleep, including complex interactions with the serotonergic system, primarily from the raphe nuclei in the medulla, which inhibit the REM generating pontine tegmentum nuclei. Clinically, patients treated with antidepressants, particularly with serotonergic properties (particularly SSRIs), tend to suppress REM sleep and may also lead to REM without atonia and/or trigger RBD events.

The melatonin MT1 and MT2 receptors likely both affect the Non-REM/REM ratio with activation of the MT2 leading to earlier and more abundant Non-REM sleep, while MT1 receptors favoring REM sleep. Furthermore, RBD is common in patients with Parkinson's disease, and a reduced number of melatonin receptors has been found in the areas involved in the neurodegeneration. A recent study found reduced MT1 receptor expression in the striatum and amygdala and reduced MT2 receptor expression in the substantia nigra and amygdala. In addition to circadian phase shift, activation of melatonin MT1 and MT2 receptors has been implicated as a potential protective mechanism against multiple other progressive neurodegenerative disorders, while MT2 receptors have been implicated in neurogenesis. Thus, REM suppression and/or disruption, as a result of the neurodegenerative process, that also involves impaired MT1 and MT2 receptor function, may be a key mechanism for RBD pathophysiology and potential therapeutic target.

### RBD Treatment

Treatment options for RBD are limited. The most commonly used agent is clonazepam, a benzodiazepine, which must be used with caution in patient with dementia symptoms and has many potentially serious side effects. Due to the strong association with neurodegenerative conditions, RBD patients are likely to have contraindications for benzodiazepine treatment. This creates a need for other medications that can be safely used in patients who are elderly and/or have neurodegenerative comorbid conditions. If the mechanism for RBD includes REM sleep disruption, improved REM sleep regulation may be useful in the treatment of RBD.

Melatonin is the most common therapeutic alternative to clonazepam for RBD. Initial studies may have been partially prompted by its high clinical convenience: a very favorable side effect profile, and availability in the US. It was first reported as effective in a case report in 1997 of a 64-year-old man who experienced improvement of his RBD symptoms after treatment with 3 mg melatonin, without any change in his REM proportion on polysomnography. Further studies have included open label case series. In one recent study, melatonin was found to be equally effective as clonazepam for RBD treatment. However, studies have been small, open label, sometimes retrospective, and generally the timing of melatonin administration is not consistently reported.

But the use of melatonin comes with a number of clinical challenges, since the medication is over the counter, not regulated, and dose and bioavailability can vary widely. As such, melatonin agonists, particularly those with higher melatonin receptor affinities than melatonin itself, may be useful in the treatment of RBD.

The use of ramelteon has been reported successful in some cases. In 2013, Nomura et al. used 8 mg of ramelteon in the treatment of two patients who had polysomnographically confirmed RBD in association with Parkinsonian syndromes. One patient had multisystem atrophy and could not tolerate clonazepam due to the liability of the patient's blood pressure. The other patient experienced persistent symptoms despite clonazepam treatment. Both patients had improvement of their RBD symptoms, including the RBD severity scale (RBDSS).

Esaki et al. treated 12 consecutive patients with idiopathic RBD in an open label trial, using 8 mg ramelteon given 30 minutes before bedtime and reported a trend towards improvement. Another study examined the effect of ramelteon on motor and non-motor symptoms in patients with Parkinson's disease, with or without RBD, and reported improvement in a variety of measures after treatment, including a statistically significant RBD improvement.

Novel data has emerged in the past two years regarding the chronotherapeutic aspects of RBD, suggesting a potential not only for improved symptom control, but also long-term benefit in terms of decreasing neurodegeneration. However, due to a large first pass effect, the mean systemic availability of ramelteon following an oral dose is less than 2% and there is a large degree of inter-subject variability in plasma concentration after exposure. Thus, another melatonin agonist could be very helpful for patients with REM behavior disorder, potentially providing a more effective treatment option for this disease and allowing safer control of the symptoms, particularly among those who cannot use benzodiazepines.

### SUMMARY

One aspect of the invention includes a method of treating a patient suffering from rapid eye movement (REM) behavior disorder (RBD) comprising: administering to the patient an effective dose of tasimelteon before bedtime.

Another aspect of the invention provides, in a method of administering tasimelteon to a patient, the improvement comprising: selecting as said patient an individual suffering from rapid eye movement (REM) behavior disorder (RBD).

Still another aspect of the invention provides a method of treating a patient suffering from rapid eye movement (REM) behavior disorder (RBD) in whom treatment with a benzodiazepine was ineffective, the method comprising: administering to the patient an effective dose of tasimelteon before bedtime.

### DETAILED DESCRIPTION

Applicant has undertaken a study to assess the effects of a single oral dose of 20 mg of tasimelteon on events of dream enactment on patients with RBD. These are measured by a daily log to allow comparison to baseline on symptoms measured by validated questionnaires (Insomnia Severity Index [ISI], Pittsburgh Sleep Quality Inventory [PSQI], Epworth Sleepiness Scale [ESS], Patient Global Impression of Change Scale (PGI-C)). Subject rest/activity patterns are also measured from actigraphy.

Further, the study assesses the effects of 20 mg tasimelteon on patients who have reduced or aberrant melatonin secretion compared to normal secretion by measuring salivary dim light melatonin onset (DLMO) at baseline and correlating this with the degree of change in RBD symptoms by end of the study.

The study also assesses any role a patient's unique genome may play in their response to tasimelteon, as obtained via whole genome sequencing.

Finally, the study assesses the safety and tolerability of a daily single oral dose of 20 mg tasimelteon.

Patients aged 18 to 85 with a diagnosis of RBD confirmed by polysomnography and without contraindications to tasimelteon or medical or psychiatric conditions that would limit their ability to participate or increase risk to are eligible for participation.

During the treatment phase, patients are administered one 20 mg tasimelteon capsule nightly for four weeks. Each patient keeps a daily sleep diary containing a diagram similar to a calendar with which the patient can track their intake of caffeine, medicine, and alcohol, if they exercised, when they went to bed, fell asleep, or took a nap, and when they woke up. All recordings are measured as the number of events and the time of day.

The daily sleep diary also contains a series of questions. Some ask the participant to record the time the diary is completed, the time they took the study drug, and the time that they think they had a dream reenactment event. Other questions are subjective yes/no questions, such as whether the patient slept the night before, whether the patient woke up naturally, and whether the patient took the dose of tasimelteon. Patients are also asked to recall how many dream enactments they had the previous night, if any.

Finally, study investigators assess participant symptoms using the Clinical Global Impression of Change Scale (CGI-C).

The terminology used herein is for the purpose of describing particular embodiments only and is not intended to be limiting of the disclosure. As used herein, the singular forms “a,” “an,” and “the” are intended to include the plural forms as well, unless expressly states otherwise or the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described element, event, or circumstance may or may not occur, and that the description includes instances where the element, event, or circumstance occurs or is present and instances where it does not occur or is not present.

The corresponding structures, materials, acts, and equivalents of all means or step plus function elements in the claims are intended to include any structure, material, or act for performing a function in combination with other claimed elements as specifically claimed. The description of the present disclosure is presented for purposes of illustration and description but is not intended to be exhaustive or limited to the disclosure in the form disclosed. Many modifications and variations will be apparent to those of ordinary skill in the art without departing from the scope and spirit of the disclosure. Any embodiments chosen and described herein appear to best explain the principles of the disclosure and their practical application, and to enable others of ordinary skill in the art to understand the disclosure for various embodiments with various modifications as are suited to the particular use contemplated.

1. A method of treating a patient suffering from rapid eye movement (REM) behavior disorder (RBD) comprising:
administering to the patient an effective dose of tasimelteon before bedtime.

2. The method of claim 1, wherein the effective dose of tasimelteon is a dose effective to alleviate at least one symptom of RBD.

3. The method of claim 1, wherein the effective dose of tasimelteon is 20 mg/day.

4. The method of claim 1, wherein the patient suffers from a neurodegenerative disorder.

5. The method of claim 4, wherein the neurodegenerative disorder is Parkinson's disease.

6. The method of claim 1, wherein the patient is one for whom treatment with a benzodiazepine is contraindicated.

7. The method of claim 1, wherein the patient is being treated with a selective serotonin reuptake inhibitor.

8. In a method of administering tasimelteon to a patient, the improvement comprising:
selecting as said patient an individual suffering from rapid eye movement (REM) behavior disorder (RBD).

9. The improvement of claim 8, wherein administering tasimelteon to the patient includes administering an effective dose of tasimelteon before bedtime.

10. The improvement of claim 9, wherein the effective dose is 20 mg/day.

11. The improvement of claim 8, wherein the patient suffers from a neurodegenerative disorder.

12. The improvement of claim 11, wherein the neurodegenerative disorder is Parkinson's disease.

13. The improvement of claim 8, wherein the patient is one for whom treatment with a benzodiazepine is contraindicated.

14. The improvement of claim 8, wherein the patient is being treated with a selective serotonin reuptake inhibitor.

15. A method of treating a patient suffering from rapid eye movement (REM) behavior disorder (RBD) in whom treatment with a benzodiazepine was ineffective, the method comprising:
administering to the patient an effective dose of tasimelteon before bedtime.

16. The method of claim 15, wherein the effective dose of tasimelteon is a dose effective to alleviate at least one symptom of RBD.

17. The method of claim 15, wherein the effective dose of tasimelteon is 20 mg/day.

18. The method of claim 15, wherein the patient suffers from a neurodegenerative disorder.

19. The method of claim 18, wherein the neurodegenerative disorder is Parkinson's disease.
